(19) United States
(12) Patent Application Publication
(10) Pub. No.: US 2015/0099927 A1
(43) Pub. Date: Apr. 9, 2015

(54) DEVICES, SYSTEMS AND METHODS FOR IMPROVED INTUBATION AND MANAGEMENT OF AIRWAYS

(71) Applicant: Ali Sadoughi, St. Louis, MO (US)

(72) Inventor: Ali Sadoughi, St. Louis, MO (US)

(21) Appl. No.: 14/506,499

(22) Filed: Oct. 3, 2014

Related U.S. Application Data
(60) Provisional application No. 61/886,634, filed on Oct. 3, 2013.

(51) Int. Cl. A61M 16/04; A61M 16/10; A61B 1/00; A61B 1/06; A61B 1/05; A61M 16/08; A61B 1/267

(52) U.S. Cl. CPC A61M 16/0488; A61M 16/0434; A61M 16/0816; A61M 16/104; A61B 1/267; A61B 1/0676; A61B 1/05; A61B 1/00052; A61M 2202/0208; A61M 2202/0241; A61M 2240/00
USPC 600/103; 128/200.26; 600/120; 600/109

(57) ABSTRACT

Devices, systems and methods for improved intubation which have particular application for awake or sedated intubation are described. An intubation scope having a handle, a flexible probe, and an articulatable tip where the handle has a control lever coupled to a rotary control wheel, which is in turn connected to control wires which when moved by the rotary control wheel cause articulation of the tip of the intubation scope. Systems utilizing the intubation scope may also include endotracheal tubes adapted to fit over the tip of the intubation scope.

### STATEMENT REGARDING FEDERALLY SPONSORED RESEARCH

N/A

### FIELD OF THE INVENTION

The present invention relates to devices and methods for aiding in the intubation of patients for mechanical ventilation and management of the airways.

### BACKGROUND OF THE INVENTION

Endotracheal intubation is a medical procedure that secures a patient's airway through the use of a tube placed in the patient's trachea to facilitate gas exchange. Endotracheal intubation is routinely performed in operating rooms or in emergency situations mostly in intensive care units or emergency rooms. Intubation is an invasive procedure often achieved with using a laryngoscope via direct visualization of the relevant physiological landmarks, however this method requires the patient to be anesthetized and sedated which in turn has negative clinical consequences, especially in severely sick patients.

In some circumstances characteristics of the patient may result in difficulties with intubation. Conditions that are associated with difficult endotracheal intubations include obesity, trauma (laryngeal fracture, mandibular or maxillary fracture, inhalation burn, cervical spine injury, temporomandibular joint dislocation), inadequate neck extension (rheumatoid arthritis, ankylosing spondylitis, halo traction), anatomic. variations (micrognathia, prognathism, large tongue, arched palate, short neck prominent upper incisors), presence of a foreign body in the upper airway, congenital anomalies (Pierre-Robin's syndrome, Treacher Collins' syndrome, laryngeal atresia, Goldenhar's syndrome, craniofacial dysostosis), infections (submandibular abscess, peritonsilar abscess, epiglottitis), tumors (cystic hygroma, hemangioma, hematoma), full stomach, or contraindications to sedative and anesthetic agents and muscle relaxants.

Patients having one or more of the characteristics associated with difficult intubations may be intubated while they are awake (“awake intubation”), in current practice, awake intubation is generally done with a flexible bronchoscope in a procedure known as flexible fiber optic intubation (“FFI”). This method is known to be a safer method of intubation, however it is often not readily available and requires an operator with special training and experience. Because of these limitations, awake intubation, although safer, may be postponed or deferred and practically utilized as a rescue method while other methods are failed; usually the encounter is an emergency situations with a rapidly crashing patient. Another shortcoming associated with FFI is delayed visualization of the larynx before intubation. Additionally, delays associated with preparation time for FFI can cause other difficulties. Currently available fiber optic scopes are long and may be difficult to manipulate. They are typically designed and constructed for use in bronchoscopy, not for endotracheal intubation. These shortcomings in the field can cause serious and life threatening complications, especially in urgent situations when FFI is considered a rescue measure.

Using the currently available fiber optic intubation techniques, it is usually difficult to find downstream airway via the mouth or nose toward the trachea. It is especially true in patients with crowded oropharynx and other similar situations

To address the shortcomings in the field, the present disclosure provides an airway management device (AMD) which can be used for awake intubation i.e. placement of a breathing tube into a patient's airway without the need for general anesthesia or other sedation. The AMD provides an easy, fast and secure Method of awake intubation. The equipment used is portable, affordable and accessible. Moreover the AMD operator requires only a short training session. The small size of the device and the portable monitor make it operator friendly and usable in a variety of clinical situations. One of the major applications of this present disclosure is a replacement for FFI.

The small size, ease of its use, and other characteristics of this device such as continuous oxygen supplementation during intubation, make it a desirable method of intubation during CPR. Using this device for securing the airway and intubating the patient is associated with a minimum interruption of cardiac compression.

In embodiments, this invention also provides a suction system to clear secretions and blood from upper airways and oral cavity, as well as a tool for removing foreign bodies from airways or other interventions on airways system, which is a complement to its function as an intubation device.

### SUMMARY OF THE INVENTION

As specified in the Background Section above, there is a need in the art to develop new therapeutic tools for securing and management of the airways, improving intubation, and particularly awake intubation.

Thus in one embodiment the invention of the present disclosure is a device for management of the airways and intubation comprising a flexible scope, the flexible scope further comprising a handle, a probe, the probe further comprising an articulatable tip, the articulatable tip further comprising a camera, a suction channel opening and a light, wherein the articulatable tip is controlled by a control mechanism. In some embodiments an endotracheal tube is removably disposed over the probe of the flexible scope.

In another embodiment the invention is a system for management of the airways and intubation comprising a flexible scope, the flexible scope further comprising a handle, a probe, the probe further comprising an articulatable tip, the articulatable tip further comprising a camera, a suction channel opening, and a light, wherein the articulatable tip is controlled by a control mechanism. In some embodiments an endotracheal tube is removably disposed over the probe of the flexible scope

In another embodiment the invention is a method of intubation comprising the steps of disposing an endotracheal tube over the probe of a flexible scope and inserting the endotracheal tube into position in a patient at the same time as the scope advances in the patient.

### BRIEF DESCRIPTION OF THE DRAWINGS

FIG. 1 shows an overall view of an embodiment of the present invention.

FIG. 2 shows an embodiment of the present invention illustrating the movement controls of the articulatable tip.

FIG. 3 shows a detailed view of the functional components of the articulatable tip and a detailed view of the functional components of the handle in the device.

### DETAILED DESCRIPTION

These and other systems, methods, objects, features, and advantages of the present disclosure will be apparent to those skilled in the art from the following detailed description of the embodiments and drawings.

All documents mentioned herein are hereby incorporated in their entirety by reference. References to items in the singular should be understood to include items in the plural, and vise versa, unless explicitly stated otherwise or clear from the text. Grammatical conjunctions are intended to express any and all disjunctive and conjunctive combinations of conjoined clauses, sentences, words, and the like, unless otherwise stated or clear from context

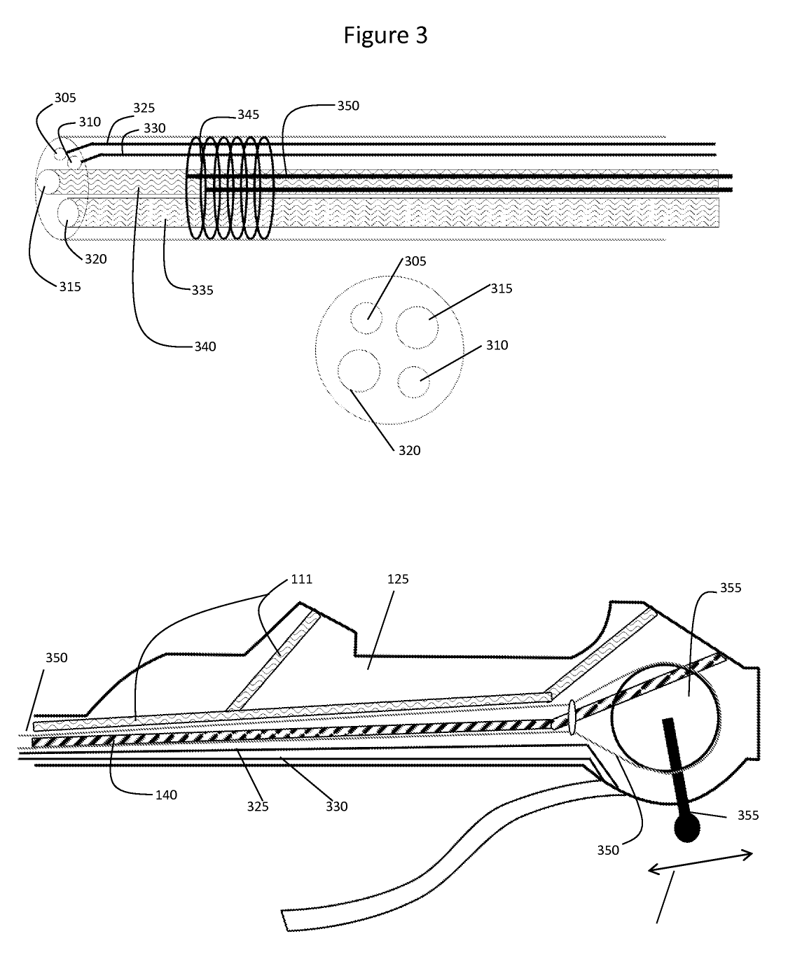

In one embodiment, the present invention is a flexible scope loaded with an endotracheal tube. The flexible scope further comprises a camera, a suction tip and a light at its tip. The scope used for the AMD is relatively short, sized to fit within an endotracheal tube. The scope is sized so that it can fit different sizes of endotracheal tubes. The largest commercially available endotracheal tube is about 35 cm long, therefore, to accommodate the use of such tubes, the length of the scope in some embodiments is about 40 cm long. These sizes are approximations of commercially available products and it is intended that the device of the present invention will vary in size to maintain compatibility with commercially available products. Therefore, in practice the scope may be from about 35 cm-55 cm long and the endotracheal tube sizes may also have similar variance. The external diameter of the probe should fit the internal diameter of different endotracheal tubes. The smallest adult endotracheal tube is size 6 (internal diameter of 6 mm). The external diameter of the probe portion of the scope may be of constant or variable diameter. The endotracheal tube is loaded onto the scope with the tip of the endotracheal tube slightly ahead of the tip of the flexible scope (˜1 cm). The tip of the scope can articulate in at least two directions. The articulation is controlled by a control mechanism on the handle of the scope (see FIG. 3). Various appropriate control mechanisms for flexible scopes of this kind are readily known to those having skill in the art. The scope may be rotated by the operator so that the tip of the scope and the endotracheal tube can be directed at any desired angle through the combination of rotation of the scope and articulation of the tip. The camera at the tip of the scope is operatively coupled to a video monitor which allows the operator to visualize physiological markers on the patient and thus guide the scope and the endotracheal tube to the desired location. The scope may further comprise one or more channels which enables suctioning and clearance of secretions which might otherwise obscure visualization of relevant physiological markers. The suction capability may also be used to prevent aspiration of secretions into the lung and thereby protect the patient from drawing those secretions into the lungs which is associated with negative clinical outcomes. The channel may also be used to administer pharmacological preparations such as anesthetizing agents, medications or supplemental oxygen. For example, the channel may be used to apply local anesthetics while placing the endotracheal tube in place in order to increase patient comfort.

In a preferred embodiment the invention of the present disclosure comprises flexible scope further comprising a handle and a probe. The probe further comprises an articulatable tip. The articulatable tip is capable of articulating in at least two directions. In some embodiments, the tip articulation is controlled by a lever on the handle of the scope or other control mechanism known to those having skill in the art. The articulatable tip further comprises a camera, a suction opening and a light. The camera and the light are controllable via a control interface on the handle of the scope. The camera is operatively coupled to a visualization device such as a video monitor. The probe of the scope further comprises a channel capable of delivering pharmacological agents to a patient and/or providing suction. An endotracheal tube is removably disposed over the probe of the scope such that the articulatable tip of the scope is located slightly back (−1 cm) from the tip of the endotracheal tube.

The flexible scope loaded with an endotracheal tube is capable of airway intubation without the need for any other instrumentation placed in the mouth or upper airway of the patient. The camera at the tip of the scope provides visualization of the airway without the need for other bulky devices such as a laryngoscope or glidescope. The light provides illumination to aid in visualization. An optional channel in the flexible scope may be used for suction to clear the airway for better visualization and to remove blood, foreign bodies, regurgitated fluids, and the like, from the airway lumen. The channel may also be used to supply supplemental oxygen and administer pharmacological agents such as local anesthetics, medications and the like. The hole for the suction/delivery channel may be placed at any location toward the tip of the flexible scope. In order to keep the cost of production at a minimum and make for easy maintenance, the channel in the flexible scope may be eliminated without affecting the operation of the present invention. The flexible scope used in the AMD is shorter than conventional flexible bronchoscopes. The length may be ˜35-40 cm which is slightly longer than the largest endotracheal tubes typically used. The shorter length of the scope used in the AMD makes it easier to be used at bedside in emergency situations. Different scope sizes for adult and pediatric uses may be provided and each is designed for use with different size endotracheal tubes. A lever, operatively coupled to the articulatable tip of the flexible scope allows an operator to articulate the tip of the scope. As the tip of the scope is articulated, it simultaneously articulates the endotracheal tube that is splinted over the scope. There is a length adjuster and locking mechanism located close to the top of the flexible scope that holds and locks the endotracheal tube onto the flexible scope in the desired position based on the size of the endotracheal tube. The locking mechanism may be any mechanism known to those having skill in the art, for example, a band which can be tightened around the probe to hold the endotracheal tube in the desired position over the probe. The locking mechanism prevents any movement of the endotracheal tube over the probe while the intubation is being performed.

The endotracheal tube is splinted over the probe of the flexible scope. Splinting the flexible scope with an endotracheal tube eliminates the need for a rigid bulky device such as a laryngoscope or glidescope. The tip of the scope remains slightly inside and back from the tip of the endotracheal tube so that the camera will not touch the airway wall while advancing the device into the airway and the vision won't be obscured. The visualization achieved by using this system allows the operator to find the downstream airway and the larynx. As the tip of the endotracheal tube stays ahead of the tip of the scope, the tip of the endotracheal tube helps to push aside soft tissue and find the airway. The endotracheal tube may be lubricated before being loaded onto flexible scope. Using the invention of the present disclosure helps the operator find the downstream airway faster than conventional FFI and advance the endotracheal tube toward the trachea.

Different types of endotracheal tubes may be used with the invention. Some endotracheal tubes are made of a softer material, while others are harder, being made from a harder plastic material. Depending on characteristics of the particular patient, such as the quantity of soft tissue in the retropharyngeal area, the width of the airway, the ease with which the scope is passed through the pharynx to find the larynx, operators may choose different endotracheal tubes to better splint the flexible scope and maneuver it into place. The harder, and more rigid the endotracheal tube, the better the splint effect. On the other hand, softer, more flexible endotracheal tubes are typically easier to use for awake intubation because of their increased maneuverability. The body of the endotracheal tube and its tip are transparent in order to facilitate image capture by the camera at the tip of the flexible scope. While it is contemplated that the body of the endotracheal tube is to be transparent, that is not a feature necessary for the operation of the invention.

Since the patient may be awake during the intubation, the patient is able to communicate until the end of the procedure. The patient only loses the ability to speak once the tube/scope passes the vocal chords. It may be important to inform the patient that he or she can communicate during the whole procedure and only at the end of the procedure, when the tube passes through the vocal cords, will they lose the ability to speak. Once the process is completed, practitioners may choose to keep patients sedated, in which case there is no need for continued use of local anesthetics. Patients may choose to be sedated during the procedure and the sedation will not affect the function of the AMD. Giving step by step instructions to the patient before the procedure will facilitate the process and serve to keep the patient comfortable, and calm by decreasing their anxiety.

To use the AMD, the operator may begin the process by applying local anesthetics, such as, for example, by spraying the local anesthetic into the mouth or nose of the patient and then to the back of the patient's throat. As the operator advances the scope towards the patient's glottis, additional local anesthetics may be administered via the channel in the flexible scope, as the scope is advanced under direct visualization into the patients vocal cords and trachea. With the use of AMD, the tip of the endotracheal tube stays ahead of the tip of the scope, and it helps to push the soft tissue aside and find the airway. Shielded by the tip of the endotracheal tube, the camera remains clean and continues to work efficiently and provides better visualization because it does not touch the wall and the surrounding tissues while passing through. Simultaneous advancement and airway intubation by the scope and endotracheal tube represents a substantial advance in the field. This technique helps the operator find the downstream airway faster and advance the endotracheal tube toward the trachea. This technique makes the AMD superior to FFI in regards to obtaining a good view of the larynx before intubation and in a faster way. The endotracheal tube on the scope works as a splint. This helps the operator push the patient's soft tissues and tongue away from the scope and easily maneuver it inside the mouth and retropharyngeal area. This method makes it faster than FFI to find the larynx. Finding the larynx and vocal cords is an important step prior to intubation of the trachea. In a situation when the tip of the endobronchial tube is close to the glottis, but the operator has difficulty advancing the endotracheal tube into the trachea, the operator may find it beneficial to advance the probe without the endotracheal tube, into the trachea first, keeping the endotracheal tube tip close to the glottis, and then slide the endotracheal tube over the probe into the trachea.

In embodiments a video monitor is operatively coupled to the camera at the tip of the flexible scope. The monitor may be any video display known to those skilled in the art. The monitor may be made in various sizes. In some embodiments the display may be attached to the flexible scope while in other embodiments, the display is located remotely and coupled to the camera by any connection known to those skilled in the art, such as, for example by a cord or wireless connection. The display may be operatively coupled to technology that would enable it to capture and save images and video clips of the procedure as it is performed.

Although the systems and devices disclosed herein have been designed for use in intubation (in awake or sedated patient), they may also be used for inspection of the pharynx, larynx and vocal cord examination. Additionally, the optional channel in the flexible scope may be used as a therapeutic device to advance different tools such as grab forceps, basket, cauterization devices, and other tools used with standard bronchoscopes for diagnostic and therapeutic purposes. Further, the AMD can be used for evaluation of swallowing and assessing the risk of aspiration.

The systems, devices and methods described herein offer a variety of advantages and benefits over the prior art including, but not limited to: (1) Minimal interruption of cardiac compression during cardio pulmonary resuscitation (“CPR”). The device of the present invention provides an intubation technique that confers substantial benefits over the current intubation techniques typically used during CPR. By using a smaller device for intubating the patient and with the increased speed of intubation, it allows the personnel performing CPR to continue cardiac compressions with little or no interruption. In current practice using intubation devices of the prior art, chest compression has to be stopped for a substantial period of time when intubation of the patient is required. (2) Continuous oxygen supplementation during intubation. In current practice, effective oxygen supplementation is usually interrupted during the time it takes to intubate the patient. This can have detrimental and dangerous outcomes for the patient due to potentially severe hypoxemia. The devices of the present invention allow for the continued high flow of oxygen through channel(s) of the devices in order to provide continuous oxygen delivery to the patients' upper airways. (3) High flow oxygen and ventilation is provided during resuscitation. As a result of the 2 previously enumerated benefits, using the devices of the present invention during resuscitation and CPR will provide substantially improved oxygenation and ventilation during resuscitation. (4) Suctioning to clear secretions and blood from upper airways and oral cavity. As previously described above, the suction channel at the tip of the probe allows for the simultaneous suction of secretions and blood from the upper airways and oral cavity. This is beneficial both because the presence of secretions and blood can obscure airway visualization and make it difficult to intubate the airway, but also because the presence of secretions and blood can be hazardous to the patient if aspirated into the lungs.

Turning now the figures:

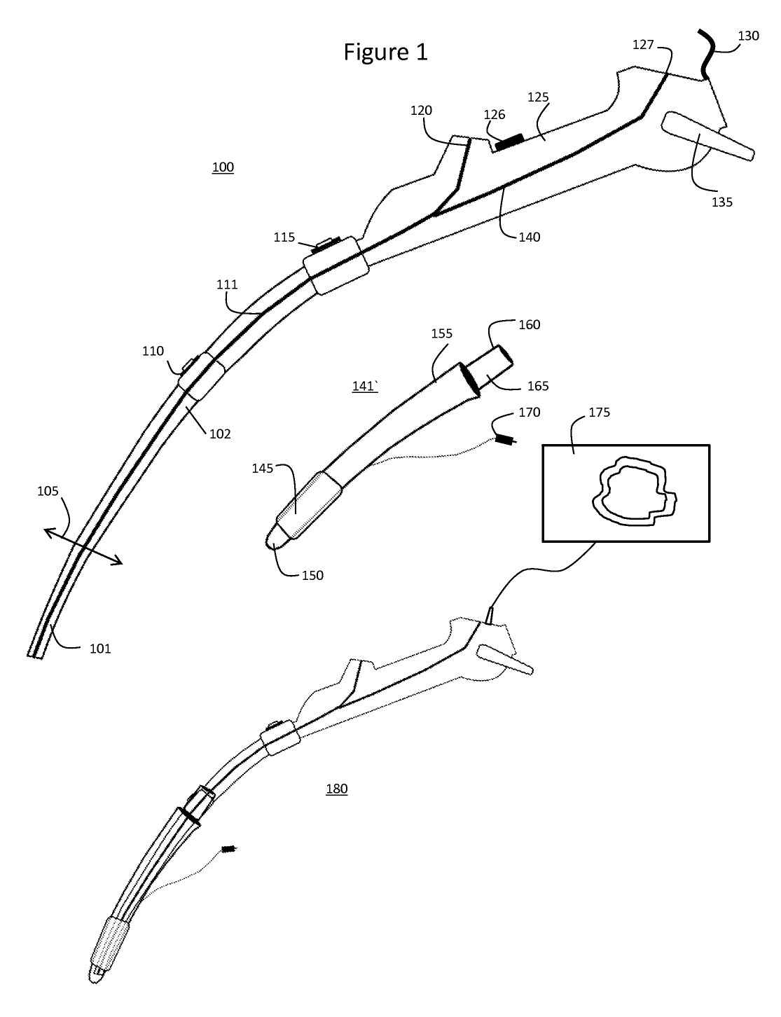

FIG. 1 shows an overall view of an embodiment of the present invention illustrating various parts of the invention. A preferred embodiment is a flexible scope (100) comprising a handle (125) and a probe (102). The probe (102) further comprises an articulatable tip (101), which articulates (105) in response to operator manipulation of a tip control mechanism, such as, for example, a control lever (135) on the handle of the scope. The articulatable tip, in some embodiments, further comprises various components such as lights, cameras, openings for suction/pharmaceutical preparation delivery, and the like. The various functions of the tip components are controlled via a control interface (126) on the handle of the scope. The probe (102) is adapted to fit inside an endotracheal tube (141) and lock the endotracheal tube in place by engaging the endotracheal tube with a locking mechanism (110). In some embodiments the scope further comprises a channel (111) or a plurality of channels that run the length of the probe (102) and handle (140). A device with a plurality, such as 2 channels will provide substantial benefits. For example, one channel can be used for oxygen supplementation while the other one is used for suction at the same time. The second channel may alternatively be used to pass different endoscopic tools that are routinely used during bronchoscopy or laryngoscopy. (including but not limited to biopsy, electro-cauterization, cryo-ablation, foreign body retrieval basket tools, and the like). A device with a single channel provides some of the advantages of the multiple channel device but is less expensive to manufacture, maintain and clean. A device without channels would be the least expensive to maintain, and manufacture. The channel(s) communicate with connections (120, 127) on the handle (125) to link the scope with suction/agent delivery apparatus. In some embodiments the scope is operatively coupled, such as by a wire (130) to a video display (175) to provide visual information to the operator. The scope is designed to operate with endotracheal tubes (141). Endotracheal tubes used in the present invention comprise a body (155), a tip (150), an inflatable cuff (145), a connection section (165), a connector (160) for attachment to a ventilator, and a balloon portion (170) operatively coupled to the cuff to monitor the pressure inside the cuff while the cuff is inflated. The endotracheal tube (141) fits over the probe portion of the scope (180).

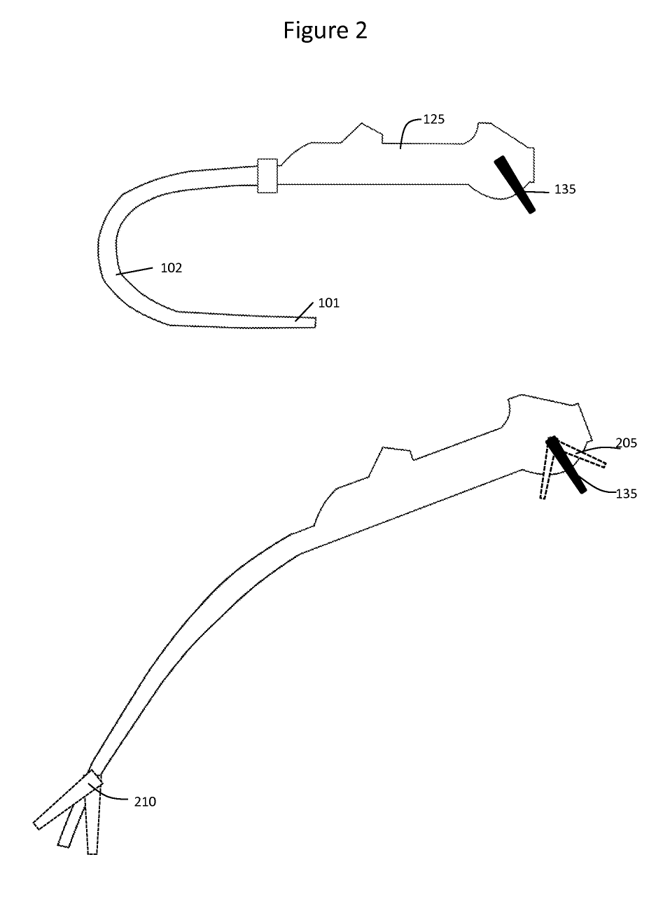

FIG. 2 shows an embodiment of the invention illustrating the movement of the articulatable tip in response to operator control. The scope of the present invention comprises a handle (125) further comprising a control lever (135) and a probe portion. The probe portion is flexible to facilitate use in patients. The control lever (135) is operatively connected to the articulatable tip (101). In response to operator manipulation (205) of the control lever (135) the articulatable tip (101) articulates in at least 2 directions (210).

FIG. 3 shows a detailed view of the various components present in some embodiments of the articulatable tip and detailed views of the components and control mechanisms in the handle. The articulatable tip, in some embodiments comprises various functional components such as channel openings (315, 320), a camera (305), and a light (310). Wired connections (330, 325) operatively connect the camera and/or light to the control interface on the handle of the scope. Channel openings (315, 320) are in communication with channels (335, 340) in order to provide suction and material delivery at the articulatable tip. In order to facilitate articulation of the articulatable tip, support structures, such as metal rings (345)are fixed to an inside surface of the articulatable tip. Control wires (350) are attached to the metal rings (345) and to a rotary control wheel (355). In use, when the operator manipulates the control lever (355), the lever turns the rotary control wheel(355) which in turn applies force to the control wires(350) which alternatively push and pull on the metal rings (345) attached to the articulatable tip which cause articulation of the articulatable tip.

In some embodiments the intubation scope will be sized for adults. In other embodiments, the intubation scope will be sized for a pediatric population.

While the present disclosure includes many embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

With respect to the above, it is to be understood that the invention is not limited in its application to the details of construction and to the arrangement of the components listed or the steps set forth in the description or illustrated in the drawings. The various apparatus and methods of the disclosed invention are capable of other embodiments, and of being practiced and carried out in various ways that would be readily known to those skilled in the art, given the present disclosure. Further, the terms and phrases used herein are for descriptive purposes and should not be construed as in any way limiting.

As such, those skilled in the art will appreciate that the conception upon which this disclosure is based may be utilized as a basis for designing other inventions with similar properties. It is important therefore that the embodiments, objects, and claims herein, be regarded as including such equivalent construction and methodology insofar as they do not depart from the spirit and scope of the present invention.

It should be noted that the components in the figures are not necessarily to scale, emphasis instead being placed upon illustrating the principles of the invention. Moreover, in the figures, like reference numerals designate corresponding parts throughout the different views. However, like parts do not always have like reference numerals. Moreover, all illustrations are intended to convey concepts, where relative sizes, shapes and other detailed attributes may be illustrated schematically rather than literally or precisely.

What is claimed is:

1. An intubation scope comprising:
a handle;
a flexible probe;
an articulatable tip;
wherein, the handle further comprises a control lever operatively coupled to a rotary control wheel, wherein a plurality of control wires are operatively coupled to the rotary control wheel such that when the control lever is manipulated, the rotary control wheel will rotate thereby moving the control wires; and
wherein the control wires extend out of the handle and through the flexible probe into the articulatable tip and connect to at least one support structure coupled to the articulatable tip such that the articulatable tip will articulate in response to manipulation of the control lever.

2. The intubation scope of claim 1 further comprising at least one channel that runs the length of the intubation scope and wherein a first channel opening is in communication with the articulatable tip and a second channel opening located on the handle of the intubation scope is in communication with a connection such that the channel can be operatively connected to a separate apparatus.

3. The intubation scope of claim 2 wherein the separate apparatus is an apparatus for providing suction.

4. The intubation scope of claim 2 wherein the separate apparatus is an apparatus for providing oxygen.

5. The intubation scope of claim 2 wherein the separate apparatus is an apparatus for providing various pharmaceutical compositions.

6. The intubation scope of claim 1 wherein the articulatable tip further comprises various functional components.

7. The intubation scope of claim 6 wherein the various functional components comprise at least one channel opening, the channel opening being in communication with a channel that runs the length of the intubation scope.

8. The intubation scope of claim 6 wherein the various functional components comprise at least one light.

9. The intubation scope of claim 6 wherein the various functional components comprise at least one camera.

10. The intubation scope of claim 6 wherein the various functional components comprise at least 1 channel opening, at least 1 camera and at least 1 light.

11. The intubation scope of claim 6 wherein the various functional components are operatively coupled to a control interface on the handle of the intubation scope.

12. The intubation scope of claim 9 further comprising a video monitor operatively coupled to the at least one camera.

13. The intubation scope of claim 1 further comprising a locking mechanism on the flexible probe to lock an endotracheal tube in place.

14. The intubation scope of claim 1 wherein the intubation scope is sized for an adult population.

15. The intubation scope of claim 1 wherein the intubation scope is sized for a pediatric population.

16. An intubation system comprising:
an intubation scope comprising:
a handle;
a flexible probe;
an articulatable tip.
wherein, the handle further comprises a control lever operatively coupled to a rotary control wheel, wherein control wires are operatively coupled to the rotary control wheel such that when the control lever is manipulated, the rotary control wheel will rotate thereby moving the control wires; and
wherein the control wires extend out of the handle and through the flexible probe into the articulatable tip and connect to at least one support structure coupled to the articulatable tip such that the articulatable tip will articulate in response to manipulation of the control lever.
an endotracheal tube comprising:
a body;
a transparent tip;
an inflatable cuff;
a connector adapted to be connected to a ventilator; and
a balloon portion operatively connected to the cuff in order to monitor the pressure inside the inflatable cuff; and
wherein the flexible probe of the intubation scope can reversibly couple to the endotracheal tube by placing the flexible probe inside the endotracheal tube.

17. The intubation system of claim 16 wherein the intubation scope further comprises a locking mechanism on the flexible probe to lock the endotracheal tube in place.
